SEQUENCE LISTING (10 sequences)

SEQ ID NO: 1 - DNA, 417 bp, synthetic construct
  source 1..417: mol_type = other DNA
atggcttatgctcagtgggttatcatcataatccacaacgtcggctcaaaggacgttaaaattgtcaatctcaaaccaagttggggtaaattacacgctgacggtgataaagacaccgaagtgtctgcatcaaagtatgaggggaccgttatcaagcccgatgaaaaactccaaatcaatgcttgcggacgttcagacgcagcagaaggaacaactggaactttcgatctagttgacccagccgacggtgataagcaagtcagacatttctactgggattgtccttggggaagcaaagctaacacttggacagtctcaggaagcaacactaaatggatgattgagtattcaggacagaatttggactctggtgcattagggacaattactgttgatactttaaaaaaaggaaactaa

SEQ ID NO: 2 - DNA, 417 bp, synthetic construct
  source 1..417: mol_type = other DNA
atggcttatgctcagtgggttatcatcataatccacaacgtcggctcaaaggacgttaaaattgtcaatctcaaaccaagttggggtaaattacacgctgacggtgataaagacaccgaagtgtctgcatcaaagtatgaggggaccgttatcagacccgatgaaaaactccaaatcaatgcttgcggacgttcagacgcagcagaaggaacaactggaactttcgatctagttgacccagccgacggtgatagacaagtcagacatttctactgggattgtccttggggaagcaaagctaacacttggacagtctcaggaagcaacactaaatggatgattgagtattcaggacagaatttggactctggtgcattagggacaattactgttgatactttaaaaaaaggaaactaa

SEQ ID NO: 3 - DNA, 516 bp, synthetic construct
  source 1..516: mol_type = other DNA
ccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgt

SEQ ID NO: 4 - DNA, 1572 bp, synthetic construct
  source 1..1572: mol_type = other DNA
atggaagccgttctatcccgtcaagctgcaactgcagaagctataggtcgttttcaagattcatccacaagcgtcggactagtagccggcagtcccagtactaggatccgtcgtcaagccgataacgtagtactcaaatccacttctcaggctggagacaccctaaacgacgtaatccaggacccaacaaggagaaataaactaataaacgacaacaacctcctaaaaggcataattatgggtcgtgacggaccagttccctcctcacgtgagttaattgtgcgaccagatacactcagggcaataattaacaacagagcaactatagaaactaccaccatggaggctgagtttaccgagacattgatggaaagcaattacaatagcgcatccgttaaggtatcagcaccatttattacagccaactccgaatatagcgagagttcctccttcaaaaacaccgagactgagaaatccatgtatacctcatccaggtatttattccctcaggggcgtattgattttaccacaccagacagcggttttgacgatgtcattaagttatctcctcagtttacaagtggagtacaagctgccttagcaaaggctaccgggactgaaaagcgtgaagccttgcaaaacttgttccaagaatatggacacgttttcagaactaaagttcacattggaggtgtcctatcagcacatactatggaaacattctctcgatccgaaaatgaaaccgaagtgaaacaagatgtcaaagctggtcttgagggtgccgtcaagggttggggaggcggggctaccgccggtcacggcaatacacaagggaccattacaacaagtcagaaccgaaaattgaatgtgaaatacatcgttaatggtggagactacactaaaatccagaatacagaagagtgggtggcatctacaaatcaaagcgagcactggcgagtaatcgaagtgaccgaagtaaccgcagttgccgatctccttccacaacccataaggggtcaagtgaaggacctattgaagccactacttgggaagtgggtcgacgtcgaaaaggttcctgggttggaatcattacctgtcagtgtctatagacccaaaggggcaatcccagcaggttggttctggcttggtgatacagcagacgcttctaaagccctcttggtcaaacctaccttacccgcccgaagcgggagaaatcccgcccttactagccttcaccagggatccggaatgactgagcagccatttgtcgatcttcctcaatatcagtacctcagcacatactttggttcatttgctcacgacaccccccctggctctactcttcgaggccttcgaccagatcatgtcttgccaggacgatatgagatgcacggtgatactatatctactgccgtatacgttactcgtcctgtggacgttccctttccagaggacgagtgttttgacttaaaatcccttgttcgtgtaaaattgcccggttccggtaatccacccaagcccaggtctgcattgaaaaaaagcatggttctttttgatagtggggaaaagtaa

SEQ ID NO: 5 - DNA, 12 bp, synthetic construct
  source 1..12: mol_type = other DNA
aaggatgaatta

SEQ ID NO: 6 - DNA, 15 bp, synthetic construct
  source 1..15: mol_type = other DNA
aagatatccatcgca

SEQ ID NO: 7 - DNA, 972 bp, synthetic construct
  source 1..972: mol_type = other DNA
aaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagc

SEQ ID NO: 8 - DNA, 102 bp, synthetic construct
  source 1..102: mol_type = other DNA
agtaatgctgctgacgaagtggctaccggtagtggcgtaaaacaaactcttaatttcgatttgttgaaactcgctggtgacgttgagagcaatccaggaccc

SEQ ID NO: 9 - DNA, 253 bp, synthetic construct
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 10 - DNA, 226 bp, synthetic construct
  source 1..226: mol_type = other DNA
cggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacct

(19) United States
(12) Patent Application Publication
(10) Pub. No.: US 2026/0297610 A1
(43) Pub. Date: Oct. 1, 2026

(54) PLANTS PRODUCING PROTEINS FOR PEST CONTROL

(71) Applicants: UNIVERZA V LJUBLJANI, Ljubljana (SI); KMETIJSKI INSTITUT SLOVENIJE, Ljubljana (SI); NACIONALNI INSTITUT ZA BIOLOGIJO, Ljubljana (SI)

(72) Inventors: Kristina Sepcic, Ljubljana (SI); Maja Grundner, Ljubljana (SI); Anastasija Panevska, Ljubljana (SI); Kristina Gruden, Ljubljana (SI); Tjasa Lukan, Ljubljana (SI); Karmen Pogacar, Ljubljana (SI); Anna Coll Rius, Ljubljana (SI); Marko Petek, Ljubljana (SI); Jaka Razinger, Ljubljana (SI); Primoz Zigon, Ljubljana (SI)

(21) Appl. No.: 19/479,699

(22) PCT Filed: Apr. 24, 2024
(86) PCT No.: PCT/EP2024/061268

§ 371 Date: Oct. 28, 2025

(30) Foreign Application Priority Data
Apr. 28, 2023 (LU) LU504095

(51) Int. Cl. C12N 15/82; C07K 14/375

(52) U.S. Cl. CPC C12N 15/8286; C07K 14/375

(57) ABSTRACT

The present invention relates to a transgenic plant or a progeny thereof which expresses or which is capable of expressing pleurotolysin A2 (PlyA2) and pleurotolysin B (PlyB), wherein said PlyB is expressed with endoplasmic reticulum localization signal, and particularly to methods and their use for controlling a plant pest, such as for controlling Colorado potato beetle (Leptinotarsa decemlineata) or Western corn rootworm (Diabrotica virgifera virgifera).

### TECHNICAL FIELD OF THE INVENTION

The present invention relates to the field of agriculture, genetic engineering and plant protection, more specifically to transgenic plants having the ability to express proteins from edible mushrooms which are toxic to certain plant pests, specifically to agricultural pest insects. More specifically, the invention relates to a transgenic plant or a progeny thereof which expresses or which is capable of expressing pleurotolysin A2 (PlyA2) and pleurotolysin B (PlyB), wherein said PlyB is expressed with endoplasmic reticulum localization signal.

### BACKGROUND

Colorado potato beetle (CPB) is one of the most important potato pests, causing high economic losses all over the world despite it is being controlled by chemical pesticides. According to FAO data for 2020, on a global scale, 16.5 million hectares were planted with potato, yielding on average 21.7 tones/hectare. The annual potato yield reaches 370 million of tones and is worth approximately 50 billion USD. Without the appropriate protection, the annual loses due to CPB would exceed 75%, which can be approximated to the economic damage of 35 billion USD.

The protection of crops against CPB is usually managed through the use of foliar insecticides that can have harmful effects on the environment and on human health. Besides that, CPB continuously develops resistance against majority of these insecticides (Alyokhin in sod. 2008). Finally, the number of approved insecticidal active ingredients decreases each year due to their disputed ecotoxicological properties. Genetically transformed potatoes expressing proteinaceous toxins Cry1Ia1 and Cry3Aa from the bacterium Bacillus thuringiensis (Bt) were introduced as an alternative to chemical insecticides in 1996; however, due to various problems associated to the emergence of resistance, changes in the marketing orientations of biotech companies and consumer aversion, these potatoes did not persist on the market (Cooper et al. 2004). Testing of new varieties of Bt potatoes is currently in course in China, where in some parts of the country CPB still holds the status of a quarantine pest, and where the production of transgenic varieties is seen as one of the most efficient solutions for the long-term reduction of crop damage caused by CPB (Mi et al. 2015). In other countries, where the use of transgenic plants is not allowed, the usual alternative to chemical insecticides comprises the use of sprays with different Bt formulations, agents based on Spinosad, plant extracts, pyrethrins, or pesticides based on RNA interference. Unfortunately, the CPB has quickly developed resistance against different Bt toxins, and also develops cross-resistance and multi-resistance against different insecticides (Cingel et al. 2016, Balaško et al. 2020). Modern agriculture therefore tends towards the discovery and development of new, environmentally and human-friendly biopesticides or plant incorporated protectants (PIPs) for CPB control.

We have recently discovered that edible mushrooms belonging to the genus Pleurotus produce protein complexes that are selectively toxic against the larvae of two important plant pests from the chrysomelid family: the Colorado potato beetle (Leptinotarsa decemlineata) and the western corn rootworm (Diabrotica virgifera virgifera) (Panevska et al., 2019). These protein complexes comprise (i) proteins of the aegerolysin family, like ostreolysin A6 (OlyA6), pleurotolysin A2 (PlyA2) and erylysin A (EryA), and (ii) proteins containing the membrane-attack complex/perforin (MACPF) domain, like pleurotolysin B (PlyB) and erylysin B (EryB). Upon ingestion, the aegerolysins bind to a specific membrane lipid receptor, ceramide phosphoethanolamine, in the membranes of insect gut epithelial cells. This step is followed by binding of the MACPF-protein partner and by the successive formation of multimeric bicomponent aegerolysin/MACPF transmembrane pores that permeabilize insect gut cell membranes and lead to insect's death (Milijaš Jotić et al., 2021). The activity of protein complexes from Pleurotus mushrooms is comparable to the activity of the Cry toxins from B. thuringiensis in the sense that they disrupt the insect's gut epithelium, however, the Cry toxins bind to membrane protein receptors and they were already introduced to CPB-resistant genetically transformed potatoes. These aegerolysin-based protein complexes are non-toxic for mammals and other non-target organisms, therefore the use of crops transformed with these proteins is considered safe for human consumption (Panevska et al., 2021).

WO2019063101 describes cytolytic bi-component protein complexes consisting of a plurality of molecules of a member of the aegerolysin family and a plurality of molecules of a member of the MACPF superfamily, and their use for controlling a plant pest.

There is a need in agriculture for improved protection of crops against pest insects, such as CPB. Specifically there is a need for such protection of crops which does not involve the spraying with insecticides and in particular which does not involve spraying with chemical insecticides.

### SUMMARY OF THE INVENTION

The present inventors have found that transgenic potatoe transformed with genes encoding plyA2 and plyB genes where PlyB is targeted to the endoplasmic reticulum (ER) facilitates improved resistance of potatoe against CPB. Such transgenic plants may in general provide solutions to e.g. agricultural crops exhibiting better resistance to plant pests and thus to enhance crop yield and minimize the use of pesticides.

Hence, in a first aspect the present invention provides a transgenic plant or a progeny thereof which expresses or which is capable of expressing pleurotolysin A2 (PlyA2) and pleurotolysin B (PlyB), wherein said PlyB is expressed with endoplasmic reticulum localization signal.

In one embodiment the PlyA2 is expressed without localization signal. In another embodiment the PlyA2 is expressed with vacuole localization signal. Without wishing to be bound by theory such different localization of PlyA2 and PlyB in the transgenic plant may contribute to minimize the possibility of their interaction and subsequent effects on the plant cells. Also, the particular localization of PlyB to the ER and localization of PlyA2 to the vacuole is believed to superior to other solutions, e.g. the other way around with PlyB to the vacuole and PlyA2 to the ER.

In a further aspect the present invention provides a method of inhibiting growth or killing a plant pest comprising expressing in a transgenic plant pleurotolysin A2 (PlyA2) and pleurotolysin B (PlyB), wherein said PlyB is expressed with endoplasmic reticulum localization signal.

In a further aspect the present invention provides a method for improving the yield of a crop in the presence of a plant pest, comprising growing the transgenic plant as described above, wherein the yield of the crop is increased in the presence of an insect pest relative to the crop not comprising said transgenic plant.

In yet a further aspect the present invention provides a method for improving the yield of a crop, comprising growing the transgenic plant as described above, wherein the yield of the crop is increased in the presence of a plant pest relative to the crop not comprising said transgenic plant.

In a yet further aspect the present invention provides the use of the transgenic plant as defined in any one of the preceding aspects and embodiments for increasing resistance against a plant pest.

In a yet further aspect the present invention provides the use of the transgenic plant as defined in any one of the preceding aspects and embodiments for protecting a plant against a plant pest.

In a yet further aspect the present invention provides the use of the transgenic plant as defined in any one of the preceding aspects and embodiments for inhibiting growth or killing a plant pest.

In a yet further aspect the present invention provides the use of the transgenic plant as defined in any one of the preceding aspects and embodiments for controlling a plant pest infestation comprising providing in the diet of the plant pest the transgenic plant of any one of the preceding aspects and embodiments or a part thereof.

The present invention can be summarized by the following items:
1. A transgenic plant or a progeny thereof which expresses or which is capable of expressing pleurotolysin A2 (PlyA2) and pleurotolysin B (PlyB), wherein said PlyB is expressed with endoplasmic reticulum localization signal.
2. The transgenic plant or a progeny thereof according to item 1, which comprises (such as stably transformed with):
   A) a polynucleotide encoding said PlyA2 and
   B) a polynucleotide encoding said PlyB and a endoplasmic reticulum localization signal sequence;
   where said polynucleotides are operably linked to at least one promoter that is functional in said transgenic plant or a progeny thereof to cause the production of mRNA molecules.
3. The transgenic plant or a progeny thereof according to any one of items 1-2, wherein said PlyA2 is expressed without localization signal.
4. The transgenic plant or a progeny thereof according to any one of items 1-2, wherein said PlyA2 is expressed with vacuole localization signal.
5. The transgenic plant or a progeny thereof according to any one of items 1-4, wherein said PlyA2 is a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 1.
6. The transgenic plant or a progeny thereof according to any one of items 1-5, wherein said PlyA2 is a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 2.
7. The transgenic plant or a progeny thereof according to any one of items 1-6, wherein said PlyA2 comprises the K85R and K52R substitutions.
8. The transgenic plant or a progeny thereof according to any one of items 1-7, wherein the expression of said PlyA2 is under control of a constitutive promoter.
9. The transgenic plant or a progeny thereof according to any one of items 1-8, wherein the expression of said PlyA2 is under control of the Cassava Vein Mosaic Virus (CsVMV) promoter, e.g. SEQ ID NO: 3.
10. The transgenic plant or a progeny thereof according to any one of items 1-9, wherein said PlyB is a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 4.
11. The transgenic plant or a progeny thereof according to any one of items 1-10, wherein said endoplasmic reticulum localization signal is SEQ ID NO: 5.
12. The transgenic plant or a progeny thereof according to item 11, wherein said localization signal is fused to the N-terminal of said PlyB.
13. The transgenic plant or a progeny thereof according to any one of items 4-12, wherein said vacuole localization signal is SEQ ID NO: 6.
14. The transgenic plant or a progeny thereof according to any one of items 1-13, wherein the expression of said PlyB is under control of a constitutive promoter.
15. The transgenic plant or a progeny thereof according to any one of items 1-14, wherein the expression of said PlyB is under control of the Cassava Vein Mosaic Virus (CsVMV) promoter, e.g. SEQ ID NO: 3.
16. The transgenic plant or a progeny thereof according to any one of items 1-14, wherein the expression of said PlyB is under control of the Petroselinum crispum ubiquitin (PcUbi) promoter, e.g. SEQ ID NO: 7.
17. The transgenic plant or a progeny thereof according to any one of items 1-16, wherein said PlyA2 is fused with a self-cleaving peptide and with PlyB in sequential orientation.
18. The transgenic plant or a progeny thereof according to item 17, wherein said self cleaving peptide is LP4/F2A, e.g. encoded by SEQ ID NO: 8
19. The transgenic plant or a progeny thereof according to any one of items 17-18, wherein said PlyA2 fused with PlyB is regulated by the CsVMV promoter.
20. The transgenic plant or progeny thereof according to any one of items 1-19, wherein said transgenic plant or progeny thereof is a crop plant.
21. The transgenic plant or progeny thereof according to any one of items 1-20, wherein said transgenic plant or progeny thereof is a potato (Solanum tuberosum) plant or maize (Zea mays) plant.
22. The transgenic plant or progeny thereof according to any one of items 1-21, wherein said transgenic plant or progeny thereof is a potato plant.
23. The transgenic plant or progeny thereof according to any one of items 1-21, wherein said transgenic plant or progeny thereof is a maize plant.
24. The transgenic plant or a progeny thereof according to any one of items 1 to 23, which has increased resistance against a plant pest.
25. The transgenic plant or a progeny thereof according to any one of items 1 to 24, wherein the plant pest is an insect.
26. The transgenic plant or a progeny thereof according to any one of items 1 to 25, wherein the plant pest is a herbivorous insect.
27. The transgenic plant or a progeny thereof according to any one of items 25-26, wherein the plant pest is a larva of the insect.
28. The transgenic plant or a progeny thereof according to item 25-26, wherein the plant pest is an imago of the insect.
29. The transgenic plant or a progeny thereof according to any one of items 25-28, wherein the insect is of the order Coleoptera.
30. The transgenic plant or a progeny thereof according to any one of items 25-29, wherein the insect is of the family Chrysomelidae.
31. The transgenic plant or a progeny thereof according to any one of items 25-30, wherein the insect is of the genus Leptinotarsa.
32. The transgenic plant or a progeny thereof according to any one of items 25-30, wherein the insect is Leptinotarsa decemlineata (Colorado potato beetle).
33. The transgenic plant or a progeny thereof according to any one of items 25-30, wherein the insect is of the genus Diabrotica.
34. The transgenic plant or a progeny thereof according to any one of items 25-30, wherein the insect is Diabrotica virgifera virgifera (Western corn rootworm).
35. The transgenic plant or a progeny thereof according to any one of items 25-30, wherein the insect is selected from the group consisting Leptinotarsa decemlineata (Colorado potato beetle) and Diabrotica virgifera virgifera (Western corn rootworm).
36. The transgenic plant or a progeny thereof according to any one of items 1 to 30, wherein the plant pest is selected from Colorado potato beetle and Western corn rootworm.
37. The transgenic plant or a progeny thereof according to any one of items 1 to 30, wherein the plant pest is Colorado potato beetle.
38. The transgenic plant or a progeny thereof according to any one of items 1 to 30, wherein the plant pest is Western corn rootworm.
39. The transgenic plant or a progeny thereof according to any one of items 1 to 38, which is a crop plant.
40. The transgenic plant or a progeny thereof according to any one of items 1 to 39, which is a potato plant.
41. The transgenic plant or a progeny thereof according to any one of items 1 to 39, which is a maize plant.
42. A method of inhibiting growth or killing a plant pest comprising expressing in a transgenic plant pleurotolysin A2 (PlyA2) and pleurotolysin B (PlyB), wherein said PlyB is expressed with endoplasmic reticulum localization signal.
43. A method for protecting a plant against a plant pest, comprising introducing a polynucleotide encoding PlyA2 and introducing a polynucleotide encoding PlyB with endoplasmic reticulum localization signal, where said polynucleotides are operably linked to at least one promoter that is functional in said plant to cause the production of mRNA molecules.
44. A method for controlling a plant pest infestation comprising providing in the diet of the pest the transgenic plant of any one of items 1-41 or a part thereof.
45. A method for improving the yield of a crop in the presence of a plant pest, comprising growing the transgenic plant of any one of items 1-41, wherein the yield of the crop is increased in the presence of an insect pest relative to the crop not comprising said transgenic plant.
46. A method for improving the yield of a crop, comprising growing the transgenic plant of any one of items 1-41, wherein the yield of the crop is increased in the presence of a plant pest relative to the crop not comprising said transgenic plant.
47. The method according to any one of items 42-46, wherein the plant pest is an insect.
48. The method according to any one of items 42-47, wherein the plant pest is a herbivorous insect.
49. The method according to any one of items 46-48, wherein the plant pest is a larva of the insect.
50. The method according to any one of items 46-48, wherein the plant pest is an imago of the insect.
51. The method according to any one of items 47-50, wherein the insect is of the order Coleoptera.
52. The method according to any one of items 47-51, wherein the insect is of the family Chrysomelidae.
53. The method according to any one of items 47-52, wherein the insect is of the genus Leptinotarsa.
54. The method according to any one of items 47-52, wherein the insect is Leptinotarsa decemlineata (Colorado potato beetle).
55. The method according to any one of items 47-52, wherein the insect is of the genus Diabrotica.
56. The method according to any one of items 46-52, wherein the insect is Diabrotica virgifera virgifera (Western corn rootworm).
57. The method according to any one of items 47-56, wherein the insect is selected from the group consisting Leptinotarsa decemlineata (Colorado potato beetle) and Diabrotica virgifera virgifera (Western corn rootworm).
58. Use of the transgenic plant as defined in any one of items 1-41 for increasing resistance against a plant pest.
59. Use of the transgenic plant as defined in any one of items 1-41 for protecting a plant against a plant pest.
60. Use of the transgenic plant as defined in any one of items 1-41 for inhibiting growth or killing a plant pest.
61. Use of the transgenic plant as defined in any one of items 1-41 for controlling a plant pest infestation comprising providing in the diet of the plant pest the transgenic plant of any one of items 1-41 or a part thereof.
62. The use according to any one of items 58-61, wherein the plant pest is an insect.
63. The use according to any one of items 58-62, wherein the plant pest is a herbivorous insect.
64. The use according to any one of items 62-63, wherein the plant pest is a larva of the insect.
65. The use according to any one of items 62-63, wherein the plant pest is an imago of the insect.
66. The use according to any one of items 62-65, wherein the insect is of the order Coleoptera.
67. The use according to any one of items 62-66, wherein the insect is of the family Chrysomelidae.
68. The use according to any one of items 62-67, wherein the insect is of the genus Leptinotarsa.
69. The use according to any one of items 62-67, wherein the insect is Leptinotarsa decemlineata (Colorado potato beetle).
70. The use according to any one of items 62-67, wherein the insect is of the genus Diabrotica.
71. The use according to any one of items 62-67 and 70, wherein the insect is Diabrotica virgifera virgifera (Western corn rootworm).
72. The use according to any one of items 62-67, wherein the insect is selected from the group consisting Leptinotarsa decemlineata (Colorado potato beetle) and Diabrotica virgifera virgifera (Western corn rootworm).

### BRIEF DESCRIPTION OF DRAWINGS

FIG. 1. Transgenic potato plants (Sphynx Protect) (left) are unharmed when attacked by Colorado potato beetle. Larval defoliation compared to non-transgenic (NT) plants having the same genetic background (NT Désirée) (right).

FIG. 2. Survival of Colorado potato beetle larvae following an 8-day exposure to transgenic plants (Sphynx Protect L1 in L2) in comparison with the non-transgenic plant (NT Désirée). Asterisk (*) denotes significant difference from control treatment (cv. Désirée).

FIG. 3. Weight of Colorado potato beetle larvae following a 10-day exposure to transgenic plants (Sphynx Protect L1 in L2) in comparison with the non-transgenic plant (NT Désirée). Different lowercase letters above bars denote significant differences between treatments (P<0.05).

FIG. 4. Average number of emerged CPB adults indicating larval metamorphosis success after exposure to transgenic plants (Sphynx Protect L1 in L2) and the non-transgenic plant (NT Désirée). Different lowercase letters above bars denote significant differences between treatments (P<0.05).

FIG. 5: Left: An overall phenotype comparison between non transgenic Désirée (A), transgenic line Sphynx Protect L1 (B) and transgenic line Sphynx Protect L2 (C). Right: Immunodetection of PlyA2 (A) and Ply B (B) in leaf extracts from transgenic lines Sphynx Protect L1 (lane 4), Sphynx Protect L2 (lane 5), non-transgenic Désirée (lane 6) and recombinant proteins (lanes 1, 2, 3:1 ng, 10 ng, 100 ng of recombinant PlyA2 or PlyB, respectively). Dashed lines indicate individual bands of the protein size standard.

FIG. 6: Left: Tuber comparison between non transgenic Desiree (A), transgenic line Sphynx Protect L1 (B) and transgenic line Sphynx Protect L2 (C). Right: Immunodetection of PlyA2 (A) and PlyB (B) in tuber extracts from potato transgenic line Sphynx Protect L1 (lanes 3A, 4A, 6B, 7B; lanes 4A and 7B: 2-times diluted sample), transgenic line Sphynx Protect L2 (lanes 5A, 6A, 8B, 9B; lanes 6A and 7B: 2-times diluted sample), non-transgenic Désirée (lanes 1A, 2A, 4B, 5B; lanes 2A and 5B: 2-times diluted sample) and recombinant proteins (lane 7A, 8A, 9A: 1 ng, 10 ng, 100 ng of PlyA2 or lane 1B, 2B, 3B: 1 ng, 10 ng, 100 ng of PlyB). Dashed lines indicate individual bands of the protein standard.

### DETAILED DESCRIPTION OF THE INVENTION

The present invention relates to the formation of cytolytic bi-component protein complexes consisting of pleurotolysin A2 (PlyA2) molecules and pleurotolysin B (PlyB) molecules in plants for controlling a plant pest, such as for controlling Colorado potato beetle (Leptinotarsa decemlineata) or Western corn rootworm (Diabrotica virgifera virgifera).

As described in the Examples, cytolytic bi-component protein complexes composed of PlyA2 and PlyB show toxic effect when ingested by invertebrates, particularly insects. This is shown in particular in transgenic plants where the PlyB is expressed with endoplasmic reticulum localization signal. These results indicate that plants expressing PlyA2 and PlyB, wherein the PlyB is expressed with endoplasmic reticulum localization signal can serve as an effective alternative biopesticide that can reduce the risk to the environment and to human health.

According to one aspect, the present invention provides a transgenic plant or a progeny thereof which expresses or which is capable of expressing pleurotolysin A2 (PlyA2) and pleurotolysin B (PlyB), wherein said PlyB is expressed with endoplasmic reticulum localization signal.

A transgenic plant or progeny thereof of the present invention suitably comprises (such as stably transformed with) one or more recombinant nucleic acid molecules (such as DNA) comprising nucleotide sequences that encode PlyA2 and PlyB as described herein, said nucleotide sequences being operably linked to at least one promoter that is functional in said plant cell to cause the production of mRNA molecules. The transgenic plant or progeny thereof may, for example, comprise one or more recombinant nucleic acid molecules (such as DNA) comprising a nucleotide sequence encoding the PlyA2, and one or more recombinant nucleic acid molecules comprising a nucleotide sequence encoding the PlyB, said nucleotide sequences being operably linked to at least one promoter that is functional in said plant cell to cause the production of mRNA molecules. The coding sequences may be comprised by the same or different recombinant nucleic acid molecules. Hence, the resulting mRNAs may be mono- or polycistronic.

The one or more recombinant nucleic acid molecules may be episomal (not contained within a chromosome) or may be stably integrated into a chromosome of the plant genome. According to certain embodiments, the one or more recombinant nucleic acid molecules may be episomal, such as in the form of a vector (such as in the form of an expression vector). According to certain embodiments, the one or more recombinant nucleic acid molecules are stably integrated into a chromosome of the plant genome.

Promoters useful in accordance with the invention are any known promoters that are functional in a plant cell to cause the production of an mRNA molecule. Many such promoters are known to the skilled person. The use of promoters for protein expression is generally known to those of skilled in the art of molecular biology, for example, see Sambrook et al., Molecular cloning: A Laboratory Manual, Cold Spring Harbor Laboratory, Cold Spring Harbor, N.Y., 1989. The promoter employed may be inducible or constitutive.

Non-limiting examples of plant functional promoters are the Lactuca sativa psbA promoter, the tobacco psbA promoter, the tobacco rrn16 PEP+NEP promoter, the CaMV 35S promoter, the 19S promoter, the tomato E8 promoter, the nos promoter, the Mac promoter, the pet E promoter or the ACT1 promoter.

The recombinant nucleic acid molecule(s) may further comprise at least one regulatory element selected from the group consisting of a 5′ untranslated region (5′ UTR), 3′ untranslated region (3′ UTR), and transit peptide region.

According to certain embodiments, the recombinant nucleic acid molecule is stably integrated into the genome of the transgenic plant or progeny thereof.

According to some embodiments, the transgenic plant or a progeny thereof comprises (such as stably transformed with):
A) a polynucleotide encoding said PlyA2 and
B) a polynucleotide encoding said PlyB and a endoplasmic reticulum localization signal sequence; where said polynucleotides are operably linked to at least one promoter that is functional in said transgenic plant or a progeny thereof to cause the production of mRNA molecules.

The PlyA2 may be expressed by the transgenic plant without a localization signal, with a localization signal or it may be expressed using a combination thereof.

According to some embodiments, the transgenic plant or a progeny thereof expresses or is capable of expressing PlyA2 without a localization signal.

According to some embodiments, the transgenic plant or a progeny thereof expresses or is capable of expressing PlyA2 with vacuole localization signal.

According to some embodiments, the transgenic plant or a progeny thereof expresses or is capable of expressing PlyA2 under control of a constitutive promoter.

According to some embodiments, the transgenic plant or a progeny thereof expresses or is capable of expressing PlyA2 is under control of the Cassava Vein Mosaic Virus (CsVMV) promoter, e.g. SEQ ID NO: 3.

Different variants of PlyA2 may be used in the present invention.

According to some embodiments, the transgenic plant or a progeny thereof expresses or is capable of expressing PlyA2 as a polypeptide comprising an amino acid sequence having at least 70%, at least 75%, at least 80%, at least 85%, at least 90%, at least 95%, at least 97%, at least 99% or 100% sequence identity with SEQ ID NO: 1.

According to some embodiments, the transgenic plant or a progeny thereof expresses or is capable of expressing PlyA2 as a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 2.

According to some embodiments, the transgenic plant or a progeny thereof expresses or is capable of expressing PlyA2 that comprises the K85R and K52R substitutions.

According to some embodiments, the transgenic plant or a progeny thereof expresses or is capable of expressing PlyB as a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 4.

According to some embodiments, the transgenic plant or a progeny thereof comprises an endoplasmic reticulum localization signal which is SEQ ID NO: 5.

According to some embodiments, the transgenic plant or a progeny thereof comprises an endoplasmic reticulum localization signal which is fused to the N-terminal of said PlyB.

According to some embodiments, the transgenic plant or a progeny thereof comprises a vacuole localization signal which is SEQ ID NO: 6.

According to some embodiments, the transgenic plant or a progeny thereof has the expression of said PlyB under control of a constitutive promoter.

According to some embodiments, the transgenic plant or a progeny thereof has the expression of said PlyB under control of the Cassava Vein Mosaic Virus (CsVMV) promoter, e.g. SEQ ID NO: 3.

According to some embodiments, the transgenic plant or a progeny thereof has the expression of said PlyB under control of the Petroselinum crispum ubiquitin (PcUbi) promoter, e.g. SEQ ID NO: 7.

According to some embodiments, the transgenic plant or a progeny thereof comprises PlyA2 which is fused with a self-cleaving peptide and with PlyB in sequential orientation.

According to some embodiments, the transgenic plant or a progeny thereof comprises a self cleaving peptide which is LP4/F2A, e.g. encoded by SEQ ID NO: 8.

According to some embodiments, the transgenic plant or a progeny thereof has PlyA2 fused with PlyB being regulated by the CsVMV promoter.

### Listing of the DNA Sequences:

PlyA2:
ATGGCTTATGCTCAGTGGGTTATCATCATAATCCACAACGTCGGCTCAAAGGACGTTAAAATTGTCAATCTCAAAC
CAAGTTGGGGTAAATTACACGCTGACGGTGATAAAGACACCGAAGTGTCTGCATCAAAGTATGAGGGGACCGTTA
TCAAGCCCGATGAAAAACTCCAAATCAATGCTTGCGGACGTTCAGACGCAGCAGAAGGAACAACTGGAACTTTCG
ATCTAGTTGACCCAGCCGACGGTGATAAGCAAGTCAGACATTTCTACTGGGATTGTCCTTGGGGAAGCAAAGCTA
ACACTTGGACAGTCTCAGGAAGCAACACTAAATGGATGATTGAGTATTCAGGACAGAATTTGGACTCTGGTGCAT
TAGGGACAATTACTGTTGATACTTTAAAAAAAGGAAACTAA.
PlyA2 incl K85R + K52R:
ATGGCTTATGCTCAGTGGGTTATCATCATAATCCACAACGTCGGCTCAAAGGACGTTAAAATTGTCAATCTCAAAC
CAAGTTGGGGTAAATTACACGCTGACGGTGATAAAGACACCGAAGTGTCTGCATCAAAGTATGAGGGGACCGTTA
TCAGACCCGATGAAAAACTCCAAATCAATGCTTGCGGACGTTCAGACGCAGCAGAAGGAACAACTGGAACTTTCG
ATCTAGTTGACCCAGCCGACGGTGATAGACAAGTCAGACATTTCTACTGGGATTGTCCTTGGGGAAGCAAAGCTA
ACACTTGGACAGTCTCAGGAAGCAACACTAAATGGATGATTGAGTATTCAGGACAGAATTTGGACTCTGGTGCAT
TAGGGACAATTACTGTTGATACTTTAAAAAAAGGAAACTAA.
CsVMV promoter:
CCAGAAGGTAATTATCCAAGATGTAGCATCAAGAATCCAATGTTTACGGGAAAAACTATGGAAGTATTATGTGAG
CTCAGCAAGAAGCAGATCAATATGCGGCACATATGCAACCTATGTTCAAAAATGAAGAATGTACAGATACAAGAT
CCTATACTGCCAGAATACGAAGAAGAATACGTAGAAATTGAAAAAGAAGAACCAGGCGAAGAAAAGAATCTTGA
AGACGTAAGCACTGACGACAACAATGAAAAGAAGAAGATAAGGTCGGTGATTGTGAAAGAGACATAGAGGACA
CATGTAAGGTGGAAAATGTAAGGGCGGAAAGTAACCTTATCACAAAGGAATCTTATCCCCCACTACTTATCCTTTT
ATATTTTTCCGTGTCATTTTTGCCCTTGAGTTTTCCTATATAAGGAACCAAGTTCGGCATTTGTGAAAACAAGAAAA
AATTTGGTGTAAGCTATTTTCTTTGAAGTACTGAGGATACAACTTCAGAGAAATTTGTAAGTTTGT.
PlyB:
ATGGAAGCCGTTCTATCCCGTCAAGCTGCAACTGCAGAAGCTATAGGTCGTTTTCAAGATTCATCCACAAGCGTCG
GACTAGTAGCCGGCAGTCCCAGTACTAGGATCCGTCGTCAAGCCGATAACGTAGTACTCAAATCCACTTCTCAGGC
TGGAGACACCCTAAACGACGTAATCCAGGACCCAACAAGGAGAAATAAACTAATAAACGACAACAACCTCCTAAA
AGGCATAATTATGGGTCGTGACGGACCAGTTCCCTCCTCACGTGAGTTAATTGTGCGACCAGATACACTCAGGGCA
ATAATTAACAACAGAGCAACTATAGAAACTACCACCATGGAGGCTGAGTTTACCGAGACATTGATGGAAAGCAAT
TACAATAGCGCATCCGTTAAGGTATCAGCACCATTTATTACAGCCAACTCCGAATATAGCGAGAGTTCCTCCTTCAA
AAACACCGAGACTGAGAAATCCATGTATACCTCATCCAGGTATTTATTCCCTCAGGGGCGTATTGATTTTACCACAC
CAGACAGCGGTTTTGACGATGTCATTAAGTTATCTCCTCAGTTTACAAGTGGAGTACAAGCTGCCTTAGCAAAGGC
TACCGGGACTGAAAAGCGTGAAGCCTTGCAAAACTTGTTCCAAGAATATGGACACGTTTTCAGAACTAAAGTTCAC
ATTGGAGGTGTCCTATCAGCACATACTATGGAAACATTCTCTCGATCCGAAAATGAAACCGAAGTGAAACAAGAT
GTCAAAGCTGGTCTTGAGGGTGCCGTCAAGGGTTGGGGAGGCGGGGCTACCGCCGGTCACGGCAATACACAAGG
GACCATTACAACAAGTCAGAACCGAAAATTGAATGTGAAATACATCGTTAATGGTGGAGACTACACTAAAATCCA
GAATACAGAAGAGTGGGTGGCATCTACAAATCAAAGCGAGCACTGGCGAGTAATCGAAGTGACCGAAGTAACCG
CAGTTGCCGATCTCCTTCCACAACCCATAAGGGGTCAAGTGAAGGACCTATTGAAGCCACTACTTGGGAAGTGGG
TCGACGTCGAAAAGGTTCCTGGGTTGGAATCATTACCTGTCAGTGTCTATAGACCCAAAGGGGCAATCCCAGCAG
GTTGGTTCTGGCTTGGTGATACAGCAGACGCTTCTAAAGCCCTCTTGGTCAAACCTACCTTACCCGCCCGAAGCGG
GAGAAATCCCGCCCTTACTAGCCTTCACCAGGGATCCGGAATGACTGAGCAGCCATTTGTCGATCTTCCTCAATATC
AGTACCTCAGCACATACTTTGGTTCATTTGCTCACGACACCCCCCCTGGCTCTACTCTTCGAGGCCTTCGACCAGAT
CATGTCTTGCCAGGACGATATGAGATGCACGGTGATACTATATCTACTGCCGTATACGTTACTCGTCCTGTGGACG
TTCCCTTTCCAGAGGACGAGTGTTTTGACTTAAAATCCCTTGTTCGTGTAAAATTGCCCGGTTCCGGTAATCCACCC
AAGCCCAGGTCTGCATTGAAAAAAAGCATGGTTCTTTTTGATAGTGGGGAAAAGTAA.
KDEL (ER localisation):
AAGGATGAATTA.
KISIA (Vacuole localisation signal:
AAGATATCCATCGCA.
PcUbi promoter:
AAAAATTACGGATATGAATATAGGCATATCCGTATCCGAATTATCCGTTTGACAGCTAGCAACGATTGTACAATTG
CTTCTTTAAAAAAGGAAGAAAGAAAGAAAGAAAAGAATCAACATCAGCGTTAACAAACGGCCCCGTTACGGCCCA
AACGGTCATATAGAGTAACGGCGTTAAGCGTTGAAAGACTCCTATCGAAATACGTAACCGCAAACGTGTCATAGT
CAGATCCCCTCTTCCTTCACCGCCTCAAACACAAAAATAATCTTCTACAGCCTATATATACAACCCCCCCTTCTATCTC
TCCTTTCTCACAATTCATCATCTTTCTTTCTCTACCCCCAATTTTAAGAAATCCTCTCTTCTCCTCTTCATTTTCAAGGT
AAATCTCTCTCTCTCTCTCTCTCTCTGTTATTCCTTGTTTTAATTAGGTATGTATTATTGCTAGTTTGTTAATCTGCTTA
TCTTATGTATGCCTTATGTGAATATCTTTATCTTGTTCATCTCATCCGTTTAGAAGCTATAAATTTGTTGATTTGACTG
TGTATCTACACGTGGTTATGTTTATATCTAATCAGATATGAATTTCTTCATATTGTTGCGTTTGTGTGTACCAATCCG
AAATCGTTGATTTTTTTCATTTAATCGTGTAGCTAATTGTACGTATACATATGGATCTACGTATCAATTGTTCATCTG
TTTGTGTTTGTATGTATACAGATCTGAAAACATCACTTCTCTCATCTGATTGTGTTGTTACATACATAGATATAGATC
TGTTATATCATTTTTTTTATTAATTGTGTATATATATATGTGCATAGATCTGGATTACATGATTGTGATTATTTACATG
ATTTTGTTATTTACGTATGTATATATGTAGATCTGGACTTTTTGGAGTTGTTGACTTGATTGTATTTGTGTGTGTATA
TGTGTGTTCTGATCTTGATATGTTATGTATGTGCAGC.
LP4/F2A:
AGTAATGCTGCTGACGAAGTGGCTACCGGTAGTGGCGTAAAACAAACTCTTAATTTCGATTTGTTGAAACTCGCTG
GTGACGTTGAGAGCAATCCAGGACCC.
NOS terminator:
GATCGTTCAAACATTTGGCAATAAAGTTTCTTAAGATTGAATCCTGTTGCCGGTCTTGCGATGATTATCATATAATT
TCTGTTGAATTACGTTAAGCATGTAATAATTAACATGTAATGCATGACGTTATTTATGAGATGGGTTTTTATGATTA
GAGTCCCGCAATTATACATTTAATACGCGATAGAAAACAAAATATAGCGCGCAAACTAGGATAAATTATCGCGCGC
GGTGTCATCTATGTTACTAGATC.
CaMV35S terminator:
CGGCCATGCTAGAGTCCGCAAAAATCACCAGTCTCTCTCTACAAATCTATCTCTCTCTATTTTTCTCCAGAATAATGT
GTGAGTAGTTCCCAGATAAGGGAATTAGGGTTCTTATAGGGTTTCGCTCATGTGTTGAGCATATAAGAAACCCTTA
GTATGTATTTGTATTTGTAAAATACTTCTATCAATAAAATTTCTAATTCCTAAAACCAAAATCCAGTGACCT.

Since the bi-component protein complex can be (or is) formed in situ in the transgenic plant it will be understood that the molecular composition and frequency may vary, meaning that different bi-component protein complexes formed of the same components may be present on the plane of the lipid bilayer varying in their molecular composition and frequency.

The transgenic plant may be any plant of interest. The transgenic plant may be an angiosperm or a gymnosperm. According to certain embodiments, the transgenic plant is an angiosperm. According to certain embodiments, the transgenic plant is a gymnosperm.

The transgenic plant may be a dicot or monocot. According to certain embodiments, the plant is a dicot. According to certain embodiment, the plant is a monocot.

The transgenic plant may be a food plant (i.e. a plant some parts of which provides food for animal or human consumption), such as fruit plant.

The transgenic plant may be a crop plant, such as a food crop plant.

According to some embodiments, the transgenic plant or progeny thereof is a crop plant.

According to some embodiments, the transgenic plant or progeny thereof is a potato (Solanum tuberosum) plant or maize (Zea mays) plant.

According to some embodiments, the transgenic plant or progeny thereof is a potato plant.

According to some embodiments, is a maize plant.

According to some embodiments, the transgenic plant or a progeny thereof has increased resistance against a plant pest.

According to some embodiments, the plant pest is an insect. According to some embodiments the plant pest is a herbivorous insect. According to some embodiments, the plant pest is a larva of the insect. According to some embodiments, the plant pest is an imago of the insect.

According to some embodiments, the insect is of the order Coleoptera.

According to some embodiments, the insect is of the family Chrysomelidae.

According to some embodiments, the insect is of the genus Leptinotarsa.

According to some embodiments, the insect is Leptinotarsa decemlineata (Colorado potato beetle).

According to some embodiments, the insect is of the genus Diabrotica.

According to some specific embodiments, the insect is Diabrotica virgifera virgifera (Western corn rootworm).

According to some specific embodiments, the insect is selected from the group consisting Leptinotarsa decemlineata (Colorado potato beetle) and Diabrotica virgifera virgifera (Western corn rootworm).

According to some specific embodiments, the plant pest is selected from Colorado potato beetle and Western corn rootworm.

According to some specific embodiments, the plant pest is Colorado potato beetle.

According to some specific embodiments, the plant pest is Western corn rootworm.

In a further aspect the present invention provides a method of inhibiting growth or killing a plant pest comprising expressing in a transgenic plant pleurotolysin A2 (PlyA2) and pleurotolysin B (PlyB), wherein said PlyB is expressed with endoplasmic reticulum localization signal.

According to some embodiments, the method for protecting a plant against a plant pest comprises introducing a polynucleotide encoding PlyA2 and introducing a polynucleotide encoding PlyB with endoplasmic reticulum localization signal, where said polynucleotides are operably linked to at least one promoter that is functional in said plant to cause the production of mRNA molecules.

According to some embodiments, the method for controlling a plant pest infestation comprises providing in the diet of the pest the transgenic plant of any one of items 1-41 or a part thereof.

In a further aspect the present invention provides a method for improving the yield of a crop in the presence of a plant pest, comprising growing the transgenic plant as described above, wherein the yield of the crop is increased in the presence of an insect pest relative to the crop not comprising said transgenic plant.

In yet a further aspect the present invention provides a method for improving the yield of a crop, comprising growing the transgenic plant as described above, wherein the yield of the crop is increased in the presence of a plant pest relative to the crop not comprising said transgenic plant.

According to some embodiments, the method comprises the plant pest being an insect.

According to some embodiments, the method comprises the plant pest being a herbivorous insect.

According to some embodiments, the method comprises the plant pest being a larva of the insect.

According to some embodiments, the method comprises the plant pest being an imago of the insect.

According to some embodiments, the method comprises the insect is of the order Coleoptera.

According to some embodiments, the method comprises the insect being of the family Chrysomelidae.

According to some embodiments, the method comprises the insect being of the genus Leptinotarsa.

According to some embodiments, the method comprises the insect being Leptinotarsa decemlineata (Colorado potato beetle).

According to some embodiments, the method comprises the insect is of the genus Diabrotica.

According to some embodiments, the method comprises the insect is Diabrotica virgifera virgifera (Western corn rootworm).

According to some embodiments, the method comprises the insect being selected from the group consisting Leptinotarsa decemlineata (Colorado potato beetle) and Diabrotica virgifera virgifera (Western corn rootworm).

In a yet further aspect the present invention provides the use of the transgenic plant as defined in any one of the preceding aspects and embodiments for increasing resistance against a plant pest.

In a yet further aspect the present invention provides the use of the transgenic plant as defined in any one of the preceding aspects and embodiments for protecting a plant against a plant pest.

In a yet further aspect the present invention provides the use of the transgenic plant as defined in any one of the preceding aspects and embodiments for inhibiting growth or killing a plant pest.

In a yet further aspect the present invention provides the use of the transgenic plant as defined in any one of the preceding aspects and embodiments for controlling a plant pest infestation comprising providing in the diet of the plant pest the transgenic plant of any one of the preceding aspects and embodiments or a part thereof.

According to some embodiments the use comprises the plant pest being an insect.

According to some embodiments the use comprises the plant pest being a herbivorous insect.

According to some embodiments the use comprises the plant pest being a larva of the insect.

According to some embodiments the use comprises the plant pest being an imago of the insect.

According to some embodiments the use comprises the insect being of the order Coleoptera.

According to some embodiments the use comprises the insect being of the family Chrysomelidae.

According to some embodiments the use comprises the insect being of the genus Leptinotarsa.

According to some embodiments the use comprises the insect being Leptinotarsa decemlineata (Colorado potato beetle).

According to some embodiments the use comprises the insect being of the genus Diabrotica.

According to some embodiments the use comprises the insect being Diabrotica virgifera virgifera (Western corn rootworm).

According to some embodiments the use comprises the insect being selected from the group consisting Leptinotarsa decemlineata (Colorado potato beetle) and Diabrotica virgifera virgifera (Western corn rootworm).

### Certain Definitions

As used herein, «controlling» a plant pest or «plant pest control» means reducing or eliminating the plant pest, such as the pest insect.

As used herein, a “pesticide” is a compound or composition used for reducing or eliminating insects harmful to cultivated plants.

As used herein, “vector” refers to a nucleic acid molecule capable of transporting another nucleic acid molecule to which it has been linked. One type of vector is a “plasmid”, which refers to a circular double stranded nucleic acid loop into which additional nucleic acid segments can be ligated. Certain vectors are capable of directing the expression of genes to which they are operatively linked. Such vectors are referred to herein as “expression vectors”. Certain other vectors are capable of facilitating the insertion of a recombinant DNA molecule into a genome of a plant. Such vectors are referred to herein as “transformation vectors”. In general, vectors of utility in recombinant nucleic acid techniques are often in the form of plasmids. In the present specification, “plasmid” and “vector” can be used interchangeably as the plasmid is the most commonly used form of a vector. Large numbers of suitable vectors are known to those of skill in the art and commercially available.

As used herein, “promoter” refers to a sequence of DNA, usually upstream (5′) of the coding region of a structural gene, which controls the expression of the coding region by providing recognition and binding sites for RNA polymerase and other factors which may be required for initiation of transcription. The selection of the promoter will depend upon the nucleic acid sequence of interest. A “promoter functional in a plant cell” refers to a “promoter” which is capable of supporting the initiation of transcription in plant cells, enabling the synthesis of an mRNA molecule.

As used herein, “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. A promoter sequence is “operably-linked” to a gene when it is in sufficient proximity to the transcription start site of a gene to regulate transcription of the gene.

“% sequence identity” of an amino acid sequence to a reference amino acid sequence, as used herein, defines the % identity calculated from the two amino acid sequences as follows: The sequences are aligned using Version 9 of the Genetic Computing Group's GAP (global alignment program), using the default BLOSUM62 matrix with a gap open penalty of −12 (for the first null of a gap) and a gap extension penalty of −4 (for each additional null in the gap). After alignment, % identity is calculated by expressing the number of matches as a percentage of the number of amino acids in the reference amino acid sequence.

Where a numerical limit or range is stated herein, the endpoints are included. Also, all values and sub ranges within a numerical limit or range are specifically included as if explicitly written out.

Having generally described this invention, a further understanding can be obtained by reference to certain specific examples, which are provided herein for purposes of illustration only, and are not intended to be limiting unless otherwise specified.

### Examples

Within the framework of the invention, the genetic code for the plyA2 and plyB genes, encoding the proteins PlyA2 and PlyB produced naturally by the oyster fungus, was optimized for expression in potatoes. The localization of the proteins was chosen based on the properties of the cell compartments and the properties of the proteins. PlyA2 was tested with two localization alternatives: I) with vacuole localization signal (amino acid sequence KISIA; Marin Viegas et al., 2017) on the C-terminus of the protein and II) without localization signal and therefore targeted to the cytoplasm. PlyB was targeted to the endoplasmic reticulum using the KDEL sequence (Dobhal et al., 2013; Song et al., 2018), fused to the N-terminus of the protein. The selection of the most appropriate promoter was based on the luminescence assay. Cassava vein mosaic virus (CsVMV; Samac et al., 2004) and Petroselinum crispum ubiquitin (PcUbi; Fauser et al., 2014) promoters were chosen as they allow constant expression level and appropriate expression strength. The plyA2 transcriptional unit under regulation of CsVMV promoter and the plyB transcriptional unit under regulation of PcUbi promoter were transcribed in the same forward direction, respectively. Based on in silico degradation analysis, two potential ubiquitination sites in the PlyA2 were eliminated by amino acid substitution (K85R & K52R). For the second construct, the modified PlyA2 was fused with a self-cleaving peptide (LP4/F2A; Wang et al., 2015; Sun et al., 2017) and with PlyB in sequential orientation, and their expression was regulated by the CsVMV promoter. With all the listed constructs, we transformed the Desiree plant genotype using the Agrobacterium-based transformation protocol optimized at National Institute of Biology of Slovenia. The produced transgenic plants are called Sphynx Protect plants.

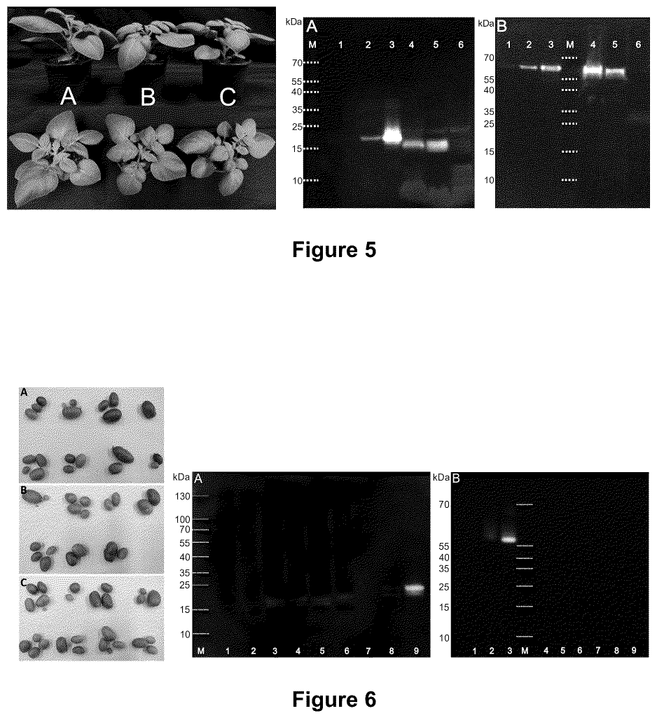

The two best performing transgenic lines, named Sphynx Protect L1 and L2, were subjected to detailed feeding trials, estimating which parameters in pest development are affected. The growth rate and overall phenotype of transgenic plants did not differ from non-transgenic (NT) Désirée plants. Western blot technique detected both proteins, PlyA2 and PlyB, in leaf extracts (FIG. 5).

When assessing the tuber production, both transgenic lines Sphynx Protect L1 and L2 had comparable or even higher tuber weight than NT Désirée plants. With western blot analysis PlyA2 protein was weakly detected in tuber extracts from Sphynx Protect plants while PlyB was not detected (FIG. 6).

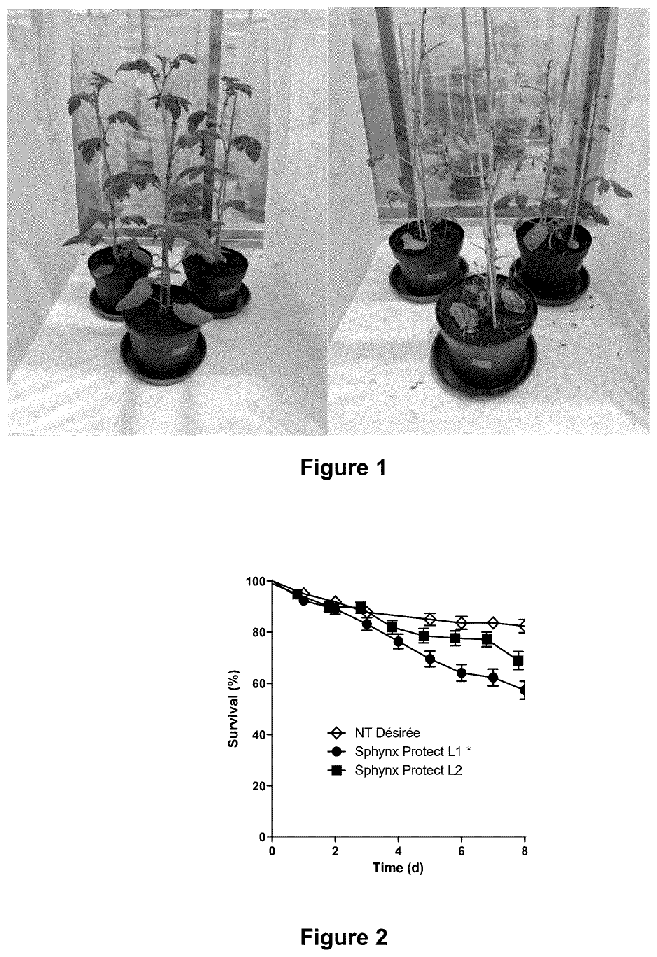

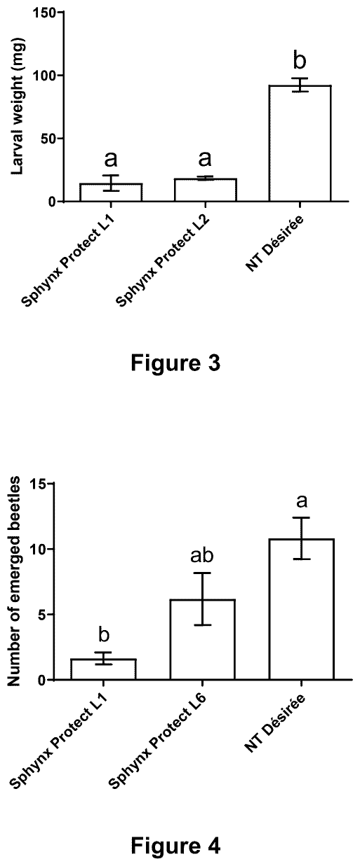

The transformed plants (FIGS. 1-4) showed a moderate toxic effect on CPB in the form of a significant increase in larval mortality in one of the Sphynx lines (Sphynx Protect L1). We observed a pronounced effect on reducing CPB larval feeding. Namely, larvae feeding on leaf tissue of Sphynx L1 and L2 were on average more than 6 and 5 times lighter, respectively, than larvae feeding on NT Désirée. Thus, even when larvae survived consumption of leaves containing toxic proteins, these larvae did not cause significant damage to protected potato plants (Sphinx). Moreover, only a fraction of the larvae that fed on Sphinx plants completed their life cycle and developed into second generation adult beetles. Thus, on average, only 11% of larvae that survived on Sphynx L1 potato plants or 37% of larvae that fed on Sphynx L2 potato plants metamorphosed into adult beetles, compared with control plants (NT Désirée), where the proportion of successfully metamorphosed larvae averaged 65%. In view of these facts, Sphinx potato plants are effectively protected from infestation by Colorado potato beetle larvae, even if they do not die immediately after consuming the tissue of the Sphinx plants.

### REFERENCES

Alyokhin, A., M. Baker M., D. Mota-Sanchez D., G. Dively G., in E. Grafius E. (2008). Colorado potato beetle resistance to insecticides. American Journal of Potato Research, 85, 395-413. Am. J. Potato Res. Cingel, A., Savić, J., Lazarević, J., Ćosić, T., Raspor, M., Smigocki, A., & Ninković, S. (2016). Extraordinary
Adaptive Plasticity of Colorado Potato Beetle: “Ten-Striped Spearman” in the Era of Biotechnological Warfare. International journal of molecular sciences, 17 (9), 1538. https://doi.org/10.3390/jims17091538
Cooper, S. G., Douches, D. S., and Grafius, E. J. (2004)., Combining genetic engineering and traditional breeding to provide elevated resistance in potatoes to Colorado potato beetle. Entomologia Experimentalis et Applicata, 112: 37-46. https://doi-org.nukweb.nuk.uni-lj.si/10.1111/1.0013-8703.2004.00182.x
Dobhal, S., Chaudhary, V. K., Singh, A., Pandey, D., Kumar, A., & Agrawal, S. (2013). Expression of recombinant antibody (single chain antibody fragment) in transgenic plant Nicotiana tabacum cv. Xanthi. Molecular Biology Reports, 40 (12), 7027-7037. https://doi.org/10.1007/s11033-013-2822-x.
Fauser, F., Schiml, S., and Puchta, H. (2014). Both CRISPR/Cas-based nucleases and nickases can be used efficiently for genome engineering in Arabidopsis thaliana. Plant Journal. 79: 348-359.
Kadoić Balaško, M.; Mikac, K. M.; Bažok, R.; Lemic, D. (2020). Modern Techniques in Colorado Potato Beetle (Leptinotarsa decemlineata Say) Control and Resistance Management: History Review and Future Perspectives. Insects, 2020, 11, 581. https://doi.org/10.3390/insects11090581
Marin Viegas, V. S.; Ocampo, C. G.; Petruccelli, S. (2017). Vacuolar deposition of recombinant proteins in plant vegetative organs as a strategy to increase yields. Bioengineered, 8, 203-211.
Mi, X., Ji, X., Yang, J., Liang, L., Si, H., Wu, J., Zhang, N., & Wang, D. (2015). Transgenic potato plants expressing cry3A gene confer resistance to Colorado potato beetle. Comptes rendus biologies, 338 (7), 443-450. https://doi.org/10.1016/j.crvi.2015.04.005
Milijaš Jotić M., Panevska A., lacovache I., Kostanjšek R., Mravinec M., Skočaj M., Zuber B., Pavšič A,. Razinger J., Modic Š., Trenti F., Guella G., Sepčić K. (2021). Dissecting Out the Molecular Mechanism of Insecticidal Activity of Ostreolysin A6/Pleurotolysin B Complexes on Western Corn Rootworm. Toxins, 13 (7), 455. https://doi.org/10.3390/toxins13070455
Panevska A., Glavan G., Jemec Kokalj A., Kukuljan V., Trobec T., Žužek M. C., Vrecl M., Drobne D., Frangež R., Sepčić K. (2021). Effects of Bioinsecticidal Aegerolysin-Based Cytolytic Complexes on Non-Target Organisms. Toxins, 13 (7), 457. https://doi.org/10.3390/toxins13070457
Panevska A., Hodnik V., Skočaj M., Novak M., Modic Š., Pavlic I. et al. (2019). Pore-forming protein complexes from Pleurotus mushrooms kill western corn rootworm and Colorado potato beetle through targeting membrane ceramide phosphoethanolamine. Scientific Reports, 25, 5073.
Samac D. A., Tesfaye M., Dornbusch M., Saruul P., Temple S. J. (2004). A comparison of constitutive promoters for expression of transgenes in alfalfa (Medicago sativa). Transgenic Research, 13 (4): 349-61. doi: 10.1023/b: trag.0000040022.84253.12. PMID: 15517994.13:349-3
Song I., Kang Y., Lee Y. K., Myung S-C., Ko K. (2018). Endoplasmic reticulum retention motif fused to recombinant anti-cancer monoclonal antibody (mAb) CO17-1A affects mAb expression and plant stress response. PLOS ONE, 13 (9): e0198978. https://doi.org/10.1371/journal.pone.0198978
Sun H., Zhou N., Wang H., Huang D., Lang Z. (2017). Processing and targeting of proteins derived from polyprotein with 2A and LP4/2A as peptide linkers in a maize expression system. PLOS One,. March 30; 12 (3): e0174804. doi: 10.1371/journal.pone.0174804. PMID: 28358924; PMCID: PMC5373624
Wang, Y., Wang, F., Wang, R., Zhao, P., and Xia, Q. (2015). 2A self-cleaving peptide-based multi-gene expression system in the silkworm Bombyx mori. Scientific ReportsSci. Rep,. 5: 16273. doi: 10.1038/srep16273

1. A transgenic plant or a progeny thereof which expresses or which is capable of expressing pleurotolysin A2 (PlyA2) and pleurotolysin B (PlyB), wherein said PlyB is expressed with endoplasmic reticulum localization signal.

2. The transgenic plant or a progeny thereof according to claim 1, which comprises (such as stably transformed with):
A) a polynucleotide encoding said PlyA2 and
B) a polynucleotide encoding said PlyB and a endoplasmic reticulum localization signal sequence;
where said polynucleotides are operably linked to at least one promoter that is functional in said transgenic plant or a progeny thereof to cause the production of mRNA molecules.

3. The transgenic plant or a progeny thereof according to claim 1, wherein said PlyA2 is expressed without localization signal.

4. The transgenic plant or a progeny thereof according to claim 1, wherein said PlyA2 is expressed with vacuole localization signal.

5. The transgenic plant or a progeny thereof according to claim 1, wherein said PlyA2 is a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 1.

6. The transgenic plant or a progeny thereof according to claim 1, wherein said PlyA2 is a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 2.

7. (canceled)

8. The transgenic plant or a progeny thereof according to claim 1, wherein the expression of said PlyA2 is under control of a constitutive promoter.

9. (canceled)

10. The transgenic plant or a progeny thereof according to claim 1, wherein said PlyB is a polypeptide comprising an amino acid sequence having at least 70%, at least 75%, at least 80%, at least 85%, at least 90%, at least 95%, at least 97%, at least 99% or 100% sequence identity with the polypeptide encoded by SEQ ID NO: 4.

11. The transgenic plant or a progeny thereof according to claim 1, wherein said endoplasmic reticulum localization signal is SEQ ID NO: 5.

12-13. (canceled)

14. The transgenic plant or a progeny thereof according to claim 1, wherein the expression of said PlyB is under control of a constitutive promoter.

15-19. (canceled)

20. The transgenic plant or progeny thereof according to claim 1, wherein said transgenic plant or progeny thereof is a crop plant.

21. The transgenic plant or progeny thereof according to claim 1, wherein said transgenic plant or progeny thereof is a potato (Solanum tuberosum) plant or maize (Zea mays) plant.

22-23. (canceled)

24. The transgenic plant or a progeny thereof according to claim 1, wherein the plant pest is an insect.

25-28. (canceled)

29. The transgenic plant or a progeny thereof according to claim 24, wherein the insect is of the family Chrysomelidae.

30. The transgenic plant or a progeny thereof according to claim 24, wherein the insect is Leptinotarsa decemlineata (Colorado potato beetle).

31. (canceled)

32. A method of inhibiting growth or killing a plant pest comprising expressing in a transgenic plant pleurotolysin A2 (PlyA2) and pleurotolysin B (PlyB), wherein said PlyB is expressed with endoplasmic reticulum localization signal.

33-36. (canceled)

37. The method according to claim 32, wherein the plant pest is an insect.

38. The method according to claim 37, wherein the insect is Leptinotarsa decemlineata (Colorado potato beetle) or Diabrotica virgifera virgifera (Western corn rootworm).

39. (canceled)

40. Use of the transgenic plant as defined in claim 1 for increasing resistance against a plant pest.

41-43. (canceled)

44. The use according to claim 40, wherein the plant pest is an insect.

45-46. (canceled)
